(19) United States
(12) Patent Application Publication
(10) Pub. No.: US 2008/0124411 A1
(43) Pub. Date: May 29, 2008

(54) Throat Spray

(75) Inventors: Jeffrey J. Fisher, Ada, MI (US); Milton W. Anderson, Eagan, MN (US)

Correspondence Address: FULBRIGHT & JAWORSKI L.L.P., 80 SOUTH EIGHTH STREET, SUITE 2100, MINNEAPOLIS, MN, 55402, US

(73) Assignee: CNS, INC., Minneapolis, MN (US)

(21) Appl. No.: 12/025,714

(22) Filed: Feb. 4, 2008

Related U.S. Application Data
(63) Continuation of application No. 10/514,375, filed on Jul. 15, 2005.
(60) Provisional application No. 60/357,787, filed on Feb. 15, 2002.

(51) Int. Cl. A61K 36/534; A61K 31/045; A61K 31/435; A61P 43/00

(52) U.S. Cl. 424/747; 514/724; 514/277

(57) ABSTRACT

An orally administered composition for relieving or eliminating snoring is described. The composition generally has four mechanisms of action for combating snoring. The composition includes a tissue-firming or astringent agent to firm up throat tissue. The composition also includes a soothing agent to soothe irritated or inflamed tissues. Also included in the composition is a lubricant to moisten dry or dehydrated tissues, and a mucous-thinning or expectorant agent to help remove any obstructive matter near the throat tissues.

### FIELD OF THE INVENTION

The present invention is directed to a composition for oral administration to the throat to relieve snoring or other irritations of the throat, by spraying or otherwise applying the composition into the back of the throat at bedtime. The composition is preferably drug-free.

### BACKGROUND

Snoring is a pernicious problem, especially for those with a regular bed partner. The noise is disruptive to sleep for both the snorer and partner, and can impair their daytime activities through sleepiness and cause relationship difficulties between them. In addition, the snorer may encounter unpleasant side effects such as dry mouth and a sore throat. Snoring is a two-part phenomenon: first, a problem such as nasal congestion causes restricted airflow through the nasal passages, leading to breathing through the mouth. Second, the turbulent flow of air through the oral passage causes vibration of the soft tissue of the palate and throat.

Current methods for treating snoring by orally administering a composition involve the use of drugs or herbal preparations, either over the counter or by prescription. As used herein, the term “drug” or the term “pharmaceutical” refers to any compound or ingredient that is intended to achieve its efficacy by absorption or ingestion into the body and that acts by metabolism or systemic means.

Descriptions of compositions for treating snoring can be found in U.S. Pat. Nos. 6,187,318; 4,668,513; and 4,556,557.

While there are several ways to address some of the problems that cause snoring, there continues to be need for a composition and method for eliminating the tissue vibration which would relieve snoring, and preferably a composition that is drug-free and free of homeopathic ingredients. This gives the user greater flexibility in using the composition of the present invention along with other treatments for treating other symptoms, such as a nasal decongestant.

The present invention is directed to reducing or eliminating the vibration of the soft tissue of the upper palate. As described previously, snoring is caused by air-turbulence induced vibrations of the soft tissue at the very back of the upper palate, which forms the beginning of the throat opening.

The present invention is based on four approaches to eliminating the soft tissue vibrations via a throat spray. First, the tissue surfaces can be firmed up using tissue-firming agents, such as an astringent. Second, the tissues can be lubricated to reduce friction-induced resonance with the turbulent airflow across them. Third, the tissues can be soothed to reduce any swelling or irritation resulting from vibration. Fourth, obstructive congesting material, such as mucous, can be thinned so that it drains from the throat, reducing turbulent flow over the obstruction and improving airflow in general. The present invention preferably encompasses all four approaches simultaneously in a single composition, but also includes the use of any of the four approaches independently or in combination with one or more of the other approaches.

The composition of the present invention may be used to treat snoring via the following method. Prior to sleeping, the composition is applied to the soft tissues of the back of the mouth and the throat, in particular to the soft palate at the rear of the mouth, to the uvula, the back of the tongue and the upper part of the pharynx. The composition is preferably applied by spray to coat those tissues, after which the user goes to sleep without disturbing the coating by eating, drinking or smoking.

### SUMMARY OF THE INVENTION

The present invention is directed to an orally administered composition for treating snoring, comprising a tissue-firming agent, a tissue-soothing agent, a tissue lubricant, and a mucous-thinning agent or an expectorant.

The present invention also includes a method for treating oral tissue to reduce or eliminate snoring, comprising the steps of firming a surface of the oral tissue; soothing the tissue; lubricating the tissue; and thinning obstructive material proximate to the tissue.

### DETAILED DESCRIPTION

The orally-administered composition of the present invention generally comprises: a tissue-firming agent in an amount ranging from between about 2.0 wt-% to about 15 wt-%; a tissue-soothing agent in an amount ranging from between about 0.1 wt-% to about 15 wt-%; a tissue lubricant in an amount ranging from between about 1.0 wt-% to about 15 wt-%; and a thinning agent in an amount ranging from between about 0.1 wt-% to about 15 wt-%. The balance of the composition is made up of an inert medium, such as water or deionized water. As used herein, the expression “wt-%” refers to weight percent, unless otherwise indicated. All the ingredients of the composition should be safe for user consumption, as the composition is preferably applied to the throat and will be absorbed by the oral tissue and swallowed by the user to some extent.

The tissue-firming agent or component useful in the present invention can be any agent that can tighten or constrict body tissues. Preferably, the tissue-firming agent is an astringent or similar compound. If an astringent is used, it is preferably selected from the group comprising alcohol, witch hazel, aluminum potassium sulfate, aluminum sodium sulfate, aluminum sulfate, zinc chloride, acacia tea, tannins, tincture of myrrh, and a combination thereof.

The tissue-soothing agent or component useful in the present invention can be any agent that can reduce irritation or inflammation of body tissues. Examples of tissue-soothing agents include, but are not limited to, essential oils, glycerin, camomile (chamomile) flowers, alpha bisabolol (an extract derived from camomile flowers), and a combination thereof.

As used herein, the term “essential oils” describes a blend of natural fragrance oils including wintergreen oil, menthol, peppermint oil, anise oil and clove oil. In addition to the essential oils mentioned herein, there are numerous other oils that would be useful in the present composition. In particular, eucalyptus, spearmint, pine, chamomile, lemon and orange oils may be useful in the composition of the present invention. In addition to, or as an alternative to using the natural essential oils of this composition, purified or synthetic versions of the essential components may be used in place of the naturally occurring oils in the present invention.

The tissue lubricant component useful in the present invention is any agent which provides moisture to body tissues, such as a humectant or a similar compound. Preferred humectants include glycerin, sorbitol, inulin, high fructose corn syrup, sucrose, phosphocholinamin, sodium alginate, and a combination thereof.

The mucous-thinning or expectorant component useful in the present invention includes any agent which thins thickened mucous and causes it to drain through nasopharyngeal passages. Preferred expectorants include essential oils, alcohol, elecampane, cayenne, cineole, and a combination thereof.

As can be seen from the foregoing, there are many ingredients available that serve more than one function in the composition of the present invention, and as described previously, the composition of the present invention may include one or more of the aforementioned components. In a particularly preferred embodiment of the present invention, the composition includes each of the four components. However, the composition is useful with just the tissue-firming component, but preferably also includes a tissue-soothing component, and more preferably also includes a tissue lubricant.

Table I shows the ranges of ingredients in one embodiment of the present invention.

TABLE I

| Ingredient | Function | High | Preferred | Low |
|---|---|---|---|---|
| Water | Medium | 50% | 86.0% | 96% |
| Alcohol | Astringent, Thinning | 15% | 7.5% | 2.0% |
| Glycerin | Soothing, Lubricant | 15% | 4.0% | 1.0% |
| Essential Oils | Soothing, Decongesting, | 10% | 0.5% | 0.1% |
| | Thinning | | | |
| Other | Stabilizers, Preservatives, etc. | 10% | 2.0% | 0% |

The essential oils useful in this invention and described in the embodiment of Table I consist primarily of a blend of menthol and wintergreen oil, preferably in approximately equal amounts, but within weight ratios of 10:1 to 1:10. Secondarily, the blend also contains peppermint oil, anise oil, and clove oil. Wintergreen and menthol preferably constitute the bulk of the blend, with each in the range of 33% to 49% of the total weight of the essential oils, with the peppermint, anise, and clove in approximately equal amounts and comprising 2% to 34% of the total weight of the essential oil blend.

In one embodiment, a bioadhesive agent is included in the composition of the present invention. The bioadhesive causes the composition to adhere to the throat tissues, and may prolong the desired effects of alleviating snoring or other throat irritations. Examples of bioadhesive agents include hydroxypropyl cellulose and carbopol.

Other ingredients, such as flavor-, appearance- or fragrance-enhancing agents, may be used as long as they do not interfere with the operation of the composition on the oral tissues. In some cases, such as with the essential oils, the components of the composition may be selected to impart a desirable flavor or fragrance to the composition. Other ingredients, such as preservatives, emulsifiers, stabilizers, and the like may be included in the composition of the present invention to enhance shelf life or use of the composition.

The composition of the present invention can be made using any conventional means to blend the components together. One preferred embodiment of the method is used to ensure that a homogeneous solution results from the blending process. Glycerin, flavorings, if any, and preservatives, if any, are added to water and blended. The essential oil and solubilizer or emulsifier, if any, are separately blended. The essential oil blend is combined with ethanol and mixed, and then blended with the glycerin-containing blend. More preferably, the final mixture is blended for an extended period of time, such as about 30 minutes, to ensure homogeneity and alcohol denaturation, if needed.

### EXAMPLE I

A clinical study was conducted with 50 patients who were self-described “nightly snorers”, were over 18 years of age, were not currently being treated for snoring, did not fit a profile for sleep apnea, and had a regular bedpartner. Daily diaries of snoring and snoring related problems were kept by both the snorer and the bedpartner. Each patient first completed the diary for a week with no treatment as a baseline, and then used each of three products in random order on subsequent weeks. The three products were all oral sprays used at bedtime per label directions. Data from the first twelve patients to complete the study were used for the analysis below.

The embodiment of the present invention tested in the clinical study had the following approximate formulation.

TABLE II
Example I Formula

| Ingredient | Function | Level |
|---|---|---|
| Water | Medium | 88.14% |
| Alcohol | Astringent, Thinning Agent | 6.90% |
| Glycerin | Soothing, Lubricant | 3.90% |
| Polysorbate 80 | Solubilizer | 0.05% |
| Sodium Saccharin | Sweetener | 0.03% |
| Cetylpyridinium Chloride | Preservative | 0.025% |
| Domiphen Bromide | Preservative | 0.005% |
| Flavor Oil | Soothing, Decongestant | 0.15% |

The flavor oil included methylsalicylate, menthol, peppermint oil, eugenol, anethol, and a propylene glycol/alcohol carrier, and was obtained from Ungerer & Company, Lincoln Park, N.J. Eugenol is the essential component of clove oil, menthol is crystallized from mentha oil, anethol is the essential component of anise oil, and methylsalicylate is the essential component of wintergreen oil.

Two other products were compared with the composition of the present invention. Product A (SnoreStop®, a product of Green Pharmaceuticals) is homeopathic and lists the following ingredients on the label: purified water, alcohol, glycerine, fructose, flavor, Nux vomica 4×, Belladonna 6×, Ephedra vulgaris 6×, Hydrastis canadensis 6×, Kali bichromicum 6×, Teucrium marum 6×, Histaminum hydrochloricum 12×.

Product B (SnoreFIX™, a product of SnoreFIX Inc.), lists the following ingredients on the label: purified water, glycerin, oat beta glucan, lecithin, DL-alphatocopheryl acetate, retinyl palmitate, ascorbic acid, linoleic acid, pyridoxine HCl, licorice extract, slippery elm extract, prickly ash extract, sweet almond oil, hybrid sunflower oil, polysorbate 20/80, eucalyptus oil, lemon oil, peppermint oil, benzyl alcohol, potassium sorbate, disodium EDTA.

An analysis of the results of the clinical study on snoring showed the following unexpected results.

### Asked of the Snorer's Bedpartner:

1. Describe your partner's snoring loudness: none, low, moderate, loud, very loud?

| | | Mean |
|---|---|---|
| Worst | No Treatment | 3.42 |
| | Product A | 3.09 |
| | Product B | 2.48 |
| Best | Current Invention | 2.30 |

2. Over how much of the night did your partner snore: none, some, half, most, all?

| | | Mean |
|---|---|---|
| Worst | No Treatment | 3.10 |
| | Product A | 2.74 |
| | Product B | 2.42 |
| Best | Current Invention | 2.14 |

3. How effective was the product in reducing your partner's snoring: extremely, very, somewhat, slightly, not at all?

| | | Mean |
|---|---|---|
| Worst | No Treatment | — |
| | Product A | 3.90 |
| | Product B | 3.05 |
| Best | Current Invention | 2.77 |

### Asked of the Snorer:

4. On awakening, was your mouth dry: no, a little, a lot?

| | | Mean |
|---|---|---|
| Worst | Product A | 1.79 |
| | Product B | 1.75 |
| | No Treatment | 1.65 |
| Best | Current Invention | 1.60 |

5. On awakening, was your throat sore: no, a little, a lot?

| | | Mean |
|---|---|---|
| Worst | Product A | 1.34 |
| | Product B | 1.30 |
| | No Treatment | 1.27 |
| Best | Current Invention | 1.23 |

6. Describe the quality of your sleep last night: excellent, good, fair, poor?

| | | Mean |
|---|---|---|
| Worst | No Treatment | 2.27 |
| | Product A | 2.04 |
| | Product B | 1.97 |
| Best | Current Invention | 1.84 |

The composition of the present invention clearly performed better than the other commercial products, and was effective at reducing snoring and snoring related symptoms.

### EXAMPLE 2

A clinical study was conducted to evaluate the efficacy of the throat spray composition of the present invention concurrently used with a Breathe Right® nasal strip which is designed to alleviate snoring and is available from CNS, Inc., Minneapolis, Minn. The study was conducted with one hundred and sixty-one (161) adult subjects. Males (90) and females (71), ranging in age from 21 to 70 years, who qualified, were recruited for this study. Recruitment was done using newspaper advertising and walk-ins to the research center. One hundred and fifty two (152) subjects completed this study. The subjects were required to be frequent snorers as reported by their bedpartners, to have a consistent bedpartner, and to not have a history or previous diagnosis of sleep apnea.

### Study Design:

One week prior to the start of this study, all subjects were instructed to refrain from the use of similar products and not to introduce any new oral or nasal products for the duration of the test. Subjects and their bedpartners were asked to complete a questionnaire as a baseline of their untreated snoring. Subjects were then randomly assigned to be given a nasal strip or throat spray to use for one week. Subjects and their bedpartners were asked to complete daily questionnaires on their snoring properties during the test period.

At the end of the first week, questionnaires and unused product were collected and both products were dispensed to the combined group for a week-long test period. Again, subjects and their bedpartners were asked to complete daily questionnaires of their snoring.

The embodiment of the present invention tested in this clinical study had the following approximate formulation.

TABLE III
Example 2 Formula

| | | Level (weight |
|---|---|---|
| Ingredient | Function | percent ± 0.01%) |
| Deionized Water | Medium | 87.69 |
| Glycerin | Soothing, Humectant | 4.00 |
| Sodium saccharin | Sweetener | 0.06 |
| Cetylpyridinium chloride | Antimicrobial | 0.05 |
| Ethanol | Astringent, Expectorant | 7.85 |
| Polysorbate 80 | Solubilizer | 0.10 |
| Flavor oils | Soothing, Expectorant | 0.25 |

The flavor oil blend used in this formula included wintergreen, menthol, peppermint, anise, and clove oils.

### Results:

The throat spray composition of the present invention is effective at reducing snoring at a statistically significant level.
Breathe Right® nasal strips are effective at reducing snoring at a statistically significant level.
The combination of nasal strips and throat spray is effective at reducing snoring at a statistically significant level.
88% of the snorers and 95% of their bedpartners considered the nasal strips effective for snoring
85% of the snorers and 95% of their bedpartners considered the throat spray effective for snoring
89% of the snorers and 97% of their bedpartners considered the combination of the two products to be effective for snoring

While the preferred application, and the one used in the clinical studies, is to spray the product onto the throat at bedtime, the formula could also be applied with a swab or other similar device, by swishing, rinsing or gargling, or from a nebulizer or humidifier. The product could also be applied during the night or any time while sleeping to prevent snoring.

While the throat spray composition tested in the clinical studies was a thin, clear liquid, there may be advantages to delivering the composition in another format, so it is retained longer on the throat. Any delivery form which permits the active ingredients to be applied onto the throat tissues is contemplated. Examples of suitable alternate delivery forms include providing the active ingredients in a lozenge or wafer, or as an edible film made with a water soluble cellulose matrix.

While not intending to be bound by theory, it is believed that the composition of the present invention relieves snoring by delaying or eliminating the onset of tissue trauma or irritation while the subject is sleeping. As such, although the product is intended to relieve snoring, the mechanism of action is generally applicable to relieve congestion and irritation of tissues, even if snoring is not present.

Although the foregoing describes many preferred embodiments of the composition and methods of use of the present invention, it is not intended to limit the true scope of the invention, which is defined by the following claims.

1. A combination for treating snoring, comprising:
a nasal strip; and
an orally administered composition comprising a tissue-firming agent, a tissue-soothing agent, a tissue lubricant, and an expectorant.

2. The combination of claim 1, wherein the tissue-firming agent of the composition is an astringent.

3. The combination of claim 2, wherein the astringent of the composition is selected from the group comprising alcohol, witch hazel, aluminum potassium sulfate, aluminum sodium sulfate, aluminum sulfate, zinc chloride, acacia tea, tannins, tincture of myrrh, and a combination thereof.

4. The combination of claim 1, wherein the tissue-soothing agent of the composition is selected from the group comprising essential oils, glycerin, chamomile flowers, alpha bisabolol, and a combination thereof.

5. The combination of claim 1, wherein the lubricant of the composition is selected from the group comprising glycerin, sorbitol, high fructose corn syrup, inulin, sucrose, phosphocholinamin, sodium alginate, and a combination thereof.

6. The combination of claim 1, wherein the expectorant of the composition is selected from the group comprising essential oils, alcohol, elecampane, cayenne, cineole, and a combination thereof.

7. The combination of claim 1, wherein the tissue-firming agent of the composition is present in an amount ranging from about 2.0% to about 15%.

8. The combination of claim 1, wherein the tissue-soothing agent of the composition is present in an amount ranging from about 0.1% to about 15%.

9. The combination of claim 1, wherein the lubricant of the composition is present in an amount ranging from about 1.0% to about 15%.

10. The combination of claim 1, wherein the expectorant of the composition is present in an amount ranging from about 0.1% to about 15%.

11. The combination of claim 1, wherein the composition further comprises a bioadhesive.

12. The combination of claim 1, wherein the composition further comprises a decongestant.

13. The combination of claim 1, wherein the composition further comprises a preservative.

14. The combination of claim 12, wherein the decongestant of the composition is present in an amount ranging from about 0.1% to about 10%.

15. The combination of claim 13, wherein the preservative of the composition is present in an amount ranging from about 0.025% to about 10%.

16. (canceled)

17. A method for reducing or eliminating snoring, comprising the steps of:
a. firming a surface of oral tissue of a user;
b. soothing the oral tissue;
c. lubricating the oral tissue;
d. thinning or expectorating obstructive material proximate to the oral tissue; and
e. applying a nasal strip to the user.

18. The method of claim 17, wherein firming the surface of the oral tissue comprises applying an astringent selected from the group comprising alcohol, witch hazel, aluminum potassium sulfate, aluminum sodium sulfate, zinc chloride, acacia tea, tannins, tincture of myrrh, and a combination thereof, to the surface of the tissue.

19. The method of claim 17, wherein soothing the oral tissue comprises applying a tissue-soothing agent selected from the group comprising essential oils, glycerin, chamomile flowers, alpha bisabolol and a combination thereof, to the surface of the tissue.

20. The method of claim 17, wherein lubricating the oral tissue comprises applying a lubricant selected from the group comprising glycerin, sorbitol, high fructose corn syrup, inulin, sucrose, phosphocholinamin, sodium alginate, and a combination thereof, to the surface of the tissue.

21. The method of claim 17, wherein expectorating obstructive material proximate to the oral tissue comprises administering an expectorant selected from the group comprising essential oils, alcohol, elecampane, cayenne, cineole, and a combination thereof, to the tissue.

22. The method of claim 17, wherein steps a-d are performed simultaneously.

23. The method of claim 17, wherein steps a-d are performed independently of each other.

24. The combination of claim 1, wherein the expectorant of the composition comprises an essential oil blend.

25. The combination of claim 24, wherein the essential oil blend comprises wintergreen oil and menthol.

26. The combination of claim 25, wherein the essential oil blend comprises from about 33% to about 49% by weight wintergreen oil and from about 33% to about 49% by weight menthol.
